(19) United States
(12) Patent Application Publication
(10) Pub. No.: US 2017/0042626 A1
(43) Pub. Date: Feb. 16, 2017

(54) METHOD AND PROBE FOR PROVIDING TACTILE FEEDBACK IN LAPAROSCOPIC SURGERY

(71) Applicant: Vladimir Egorov, Princeton, NJ (US)

(72) Inventor: Vladimir Egorov, Princeton, NJ (US)

(73) Assignee: Advanced Tactile Imaging, Inc., Trenton, NJ (US)

(21) Appl. No.: 15/219,536

(22) Filed: Jul. 26, 2016

Related U.S. Application Data
(60) Provisional application No. 62/199,899, filed on Jul. 31, 2015.

(51) Int. Cl. A61B 34/00; A61B 1/313; A61B 1/05

(52) U.S. Cl. CPC A61B 34/76; A61B 1/05; A61B 1/3132

(57) ABSTRACT

Laparoscopic tactile imaging probe equipped with pressure sensors is configured for placement into a body cavity to provide real time tactile feedback for a region of interest during a surgical procedure. A method for providing tactile feedback in laparoscopic surgery by calculating a two-dimensional tactile feedback image using a three-dimensional tactile image, and imposing said tactile feedback image on said region of interest for review in real time by a surgeon is disclosed. A laparoscopic tactile imaging probe consists of a probe housing with a plurality of tactile sensors are located over a cylinder surface and a visual markers over said probe housing for visual acquiring probe positioning data during said deformation by a laparoscopic video camera.

### CROSS-REFERENCE TO RELATED APPLICATIONS

This patent application claims a priority benefit from the U.S. Provisional Patent Application No. 62/199,899 filed Jul. 31, 2015 by the same inventor and with the same title.

### GOVERNMENT-SUPPORTED RESEARCH

This invention was made with the US Government support under grant No. R43HD086907 awarded by the Eunice Kennedy Shriver National Institute of Child Health & Human Development of the National Institutes of Health. The Government has certain rights in this invention.

### FIELD OF THE INVENTION

The present invention generally relates to tissue characterization methods during a surgery. Specifically, the invention describes methods and devices for providing tactile feedback or tissue elasticity imaging during inpatient laparoscopic surgery.

### BACKGROUND OF THE INVENTION

The laparoscopy includes traditional laparoscopic approaches and robotic surgery systems operated through a console located away from the patient. During a laparoscopic surgery the video camera becomes a surgeon's eyes, since the surgeon uses the image from the video camera positioned inside the patient's body to perform the procedure. Visual feedback is either similar or often superior to open procedures. The robotic platform (e.g., da Vinci Surgical System) allows laparoscopic surgeons to perform more complex procedures with improved accuracy. In general gynecology and reproductive gynecology, the robot is being increasingly used for procedures such as hysterectomies, myomectomies, adnexal surgery, and tubal anastomosis. In urogynecology the robot is being utilized for sacrocolpopexy.

There is a friction between trocar ports and the laparoscopic instruments which makes sensing of tissue feedback under applied force through the instrument's shaft extremely difficult in traditional laparoscopy. Without tactile feedback, a robotic surgeon is unable to appreciate differences in tissue texture or stiffness. Without palpation access as in open surgery, the surgeon needs an additional technology which can be incorporated into the laparoscopic surgical procedure, allowing for precise real time intraoperative tumor localization that will guide the extent of surgical resection.

In the cases of sacrocolpopexy, often performed in conjunction with a hysterectomy, one of the most frustrating elements is the lack of tactile feedback at the level of the sacral promontory. The surgeon needs to open up the peritoneum and dissect the underlying tissue to expose the sacral bony structures and ligament without injury to the surrounding bowel or vascular structures. Sometimes, the sacral promontory is covered with a fat pad which makes it difficult to visualize the promontory and the “safe” space below the aorta and between the iliac arteries/veins is difficult to discern. A difference that is very easy to appreciate with an open procedure where one can easily palpate through the fatty tissues. It is that step that is very critical for the repair and that has the greatest potential for catastrophic error.

This limitation of laparoscopic surgery has been known for a long time and multiple approaches were explored to provide the tactile sensation [Okamura A M. Haptic feedback in robot-assisted minimally invasive surgery. Curr Opin Urol. 2009; 19(1): 102-7.]. However, to date, to the best of our knowledge, there is no satisfactory solution providing tactile feedback for laparoscopic surgery. Creative solutions are needed to develop compelling tactile feedback.

In the last decade, a new modality for tissue characterization termed Elasticity Imaging or

Elastography has emerged. Tactile Imaging, a branch of Elasticity Imaging, yields a tissue elasticity map, similarly to other elastographic techniques. At the same time, Tactile Imaging, which is also called “stress imaging”, “computerized palpation” or “mechanical imaging” [Sarvazyan A. Mechanical imaging: a new technology for medical diagnostics. Int. J. Med. Inform. 1998, 49(2): 195-216], most closely mimics manual palpation, because the Tactile Imaging probe with a pressure sensor array mounted on its face acts similarly to human fingers during clinical examination, slightly compressing soft tissue by the probe and detecting resulting changes in the pressure pattern.

Tactile Imaging is a medical imaging modality that translates the sense of touch into a digital image. The tactile image is a function of P(x,y,z), where P is the pressure on soft tissue surface under applied deformation and x,y,z are coordinates where pressure P was measured. The tactile image is a pressure map on which the direction of tissue deformation must be specified [van Raalte H. Egorov V. Tactile imaging markers to characterize female pelvic floor conditions. Open Journal of Obstetrics and Gynecology 2015; 5: 505-515].

Earlier we proposed tactile imaging solutions for breast, prostate and vagina [Sarvazyan A, Egorov V. Apparatus and method for mechanical imaging of breast. U.S. Pat. No. 6,620,115, Sep. 16, 2003; Sarvazyan A P, Egorov V. Device for palpation and mechanical imaging of the prostate. U.S. Pat. No. 6,142,959, Nov. 7, 2000; Egorov V, van Raalte H, Sarvazyan A P. Methods for assessment of pelvic organ conditions affecting the vagina. U.S. Pat. No. 8,187,208, May 29, 2012]. In this invention we disclose the method and device for providing tactile feedback in laparoscopic surgery. It will allow the tactile perception in both traditional laparoscopy and robotic surgery systems. The real time fusion of video stream from laparoscope with the tactile sensation data for a region of interest will increase the surgical accuracy and extend the technical capability of the laparoscopic surgery. The benefits to surgeons, patients and society are high because of the large number of performed inpatient laparoscopic surgeries which are increasing from year to year. The improved quality of surgical procedures will reduce the negative psychological impact on patients and lower the actual costs of subsequent evaluations or surgical repairs.

### SUMMARY OF THE INVENTION

The objective of the present invention is to overcome the drawbacks of the prior art and to provide novel method and device for objective characterizing a tissue or an organ during a surgery, including a laparoscopic surgery, in particular by measuring a tactile feedback from a surface of a tissue or an organ using tactile sensors mounted on a laparoscopic tactile imaging probe.

Another objective of the invention is to provide methods and devices for objective visualization and detection of elasticity boundaries for a connective tissue, an organ, an artery, a vein, a nodule, a lump, diseased or abnormal zones.

Another objective of the invention is to provide methods and devices for objective visualization and detection of a blood vessel location and boundary by detecting a pressure in blood flow and/or a pulse generated during the heartbeat.

Another objective of the invention is to provide methods and devices for objective quantitative characterization of said tactile feedback in real time.

Another objective of the invention is to overcome the greatest limitation to minimally invasive approaches is the impairment (traditional laparoscopic) or complete lack of tactile sensation (robotic) normally used to assist in surgical dissection and decision making.

A further objective of the invention is to provide methods and devices to appreciate differences in tissue texture and stiffness. Without palpation access as in open surgery, the surgeon needs an affordable imaging technology which can be incorporated into the laparoscopic surgical procedure, allowing for precise real time intraoperative tumor localization that will guide the extent of surgical resection.

A further yet objective of the invention is to provide methods and devices for objective diagnosis of a disease by comparing a calculated tactile feedback for a particular patient against respective normal values obtained from clinical data collected from a number of patients with known clinical status.

In embodiments, the method for providing tactile feedback in laparoscopic surgery comprises the steps of:

(a) inserting a tactile imaging probe into a body cavity;

(b) selecting a region of interest on a surface of a tissue or an organ by means of an image projected by a laparoscopic video camera;

(c) applying a tissue deformation to said surface by means of said probe;

(d) recording a tactile response from said surface during its deformation,

(e) acquiring probe positioning data during said tissue deformation;

(f) composing a three-dimensional tactile image using said tactile response and said probe positioning data;

(g) calculating a two-dimensional tactile feedback image using said three-dimensional tactile image; and

(h) imposing said tactile feedback image on said image of interest for review in real time by a surgeon.

In embodiments, a probe for providing tactile feedback in laparoscopic surgery comprises:

a probe housing with a front portion suitably shaped for atraumatic insertion into a body cavity,

a plurality of tactile sensors forming together a tactile array, said plurality of tactile sensors are located over a cylinder surface of said front portion, said plurality of tactile sensors are configured to record a tactile response from selected region of interest on a surface of a tissue or an organ being under a deformation provided by said front portion, and

a visual markers over said probe housing for visual acquiring probe positioning data during said deformation by a laparoscopic video camera.

### BRIEF DESCRIPTION OF DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 presents an embodiment of laparoscopic tactile imaging probe;

FIG. 2 presents a schematic diagram of an embodiment of a laparoscopic device which comprises a laparoscopic tactile imaging probe with an external data processor;

FIG. 3 illustrates the use of laparoscopic tactile imaging probe during a surgery;

FIG. 4 illustrates the use of the probe and the method for providing tactile feedback during laparoscopic surgery from the point of view of a surgeon.

FIG. 5 is a flow chart illustrating one method for providing tactile feedback during laparoscopic surgery;

FIG. 6 is a flow chart illustrating details of data processing algorithm for providing tactile feedback for method shown in FIG. 5;

FIG. 7 is a flow chart illustrating another method for providing tactile feedback during laparoscopic surgery, herein probe motion tracking data are acquired from a motion tracking sensor;

FIG. 8 is a flow chart illustrating one method for providing tactile feedback during detection of a blood vessel by measuring the pressure or a pulse from a heartbeat.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

Specific terms are used in the following description, which are defined as follows: “tactile sensor” is the sensor capable to measure an applied orthogonal force averaged per sensor area or pressure. “Tissue deformation” is used to describe soft tissue, structure and organ deformation.

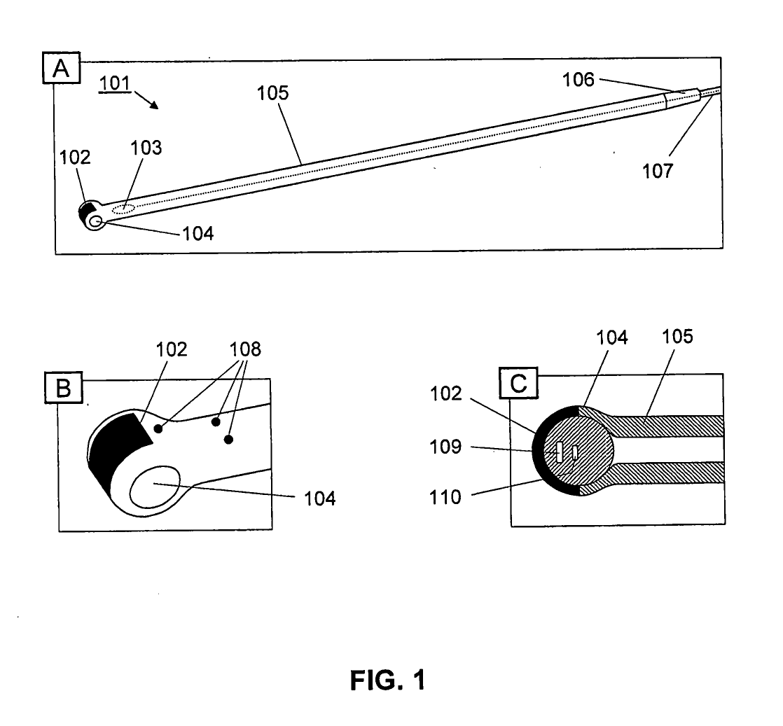

FIG. 1 presents an embodiment of laparoscopic tactile imaging probe. A probe 101 for providing tactile feedback in laparoscopic surgery consists of a probe housing with a front portion/scanhead 104 suitably shaped for atraumatic insertion into a body cavity, a plurality of tactile sensors forming together a tactile array 102, said plurality of tactile sensors are located over a cylinder surface of said front portion, said plurality of tactile sensors are configured to record a tactile response from selected region of interest on a surface of a tissue or an organ being under a deformation provided by said front portion, and a visual markers 108 over said probe housing for visual acquiring probe positioning data during said deformation by a laparoscopic video camera

In more details, FIG. 1A shows the tactile imaging probe 101 which includes a scanhead 104, a shaft 105 which can be used as a handle to manipulate the tactile imaging probe. Additionally, the probe 101 may include a motion tracking sensor 103 which might be placed inside the shaft 105 as well as in the scanhead 104. The motion tracking sensor provides three degrees of freedom (three angles or three coordinates) as well as six degrees of freedom (three angles and three coordinates) motion tracking data for the probe to be used for calculating three-dimensional coordinates for each tactile sensor during probe manipulation. The motion tracking sensor used with the probe 101 may include accelerometers, magnetometers, gyroscopes, an electromagnetic as well as an ultrasound tracker.

FIG. 1B presents a perspective view of the probe scanhead 104 with visual markers 108 over a housing of the probe for visual acquisition of probe positioning data by a laparoscopic video camera during tissue deformation. The visual markers includes at least three color points at vertices of a triangular wherein one vertex is placed on a probe shaft 105 within the visual detection by laparoscopic video camera.

FIG. 1C presents a cross-section of the probe scanhead 104 with a part of the shaft 105. Based on possibility of a surgical port used in laparoscopy and the experience gained during development and testing of various probes designed for tissue imaging, we estimated that a scanhead transversal size of about 10 mm with a curvature radius of about 5 mm would be close to optimal for the laparoscopic application. The tactile sensor array 102 may have 24 (4 by 6) tactile sensors with grid 2.0 mm by 2.0 mm. The sensor array can be assembled on a cylindrical body (see cylinder within the scanhead in FIG. 1C) with diameter about 8 mm and length of about 7.0 mm. Sensor array temperature will be maintained at 36±0.5° C. with the use of micro-heather 109 and temperature sensor 108 (FIG. 1C) to eliminate the temperature dependence of tactile sensor readings. The expected pressure response range from 20 Pa to 10,000 Pa. The tactile sensor array can be built with resistive, capacitive, optical as well as piezoresistive transducers of applied pressure to an electrical signal.

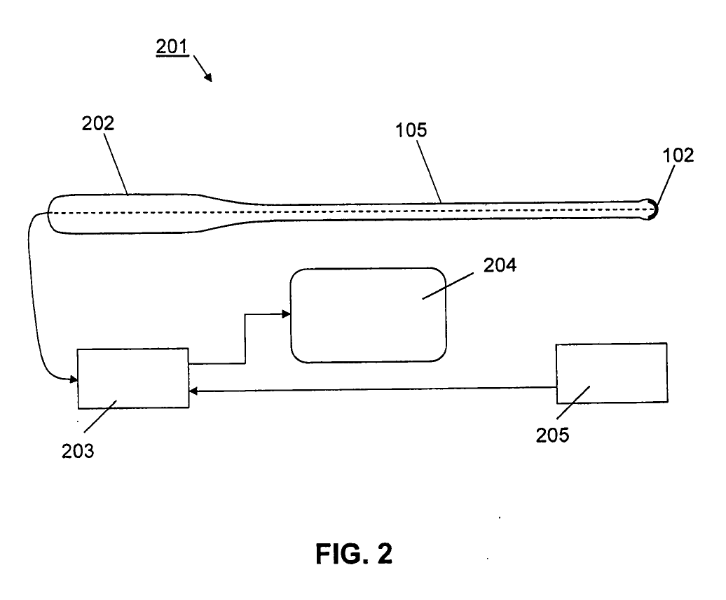

FIG. 2 presents a schematic diagram of an embodiment of a laparoscopic device 201 which comprises the laparoscopic tactile imaging probe 101, as described in FIG. 1, a data processor 203 and a display 204. A video stream from a laparoscopic video camera 205 is acquired by the data processor 203 with the purposes:
a) to calculate locations of tactile sensor array 102 within laparoscopic video frames,
b) to provide probe motion tracking data,
c) to calculate three-dimensional tactile image for a region of interest,
d) to calculate two-dimensional tactile feedback image,
e) to impose the tactile feedback image on streaming video frames,
f) to render the video stream with the imposed tactile feedback image on display 204.
   The tactile imaging probe may have a probe handle 202 for the tactile imaging probe manipulation.

Another embodiment of a laparoscopic device (not shown in FIGS) comprises the laparoscopic tactile imaging probe 101, as described in FIG. 1 and the data processor 203, as described in FIG. 2. The data processor renders the video stream with the imposed tactile feedback image on a laparoscopic display during a surgery.

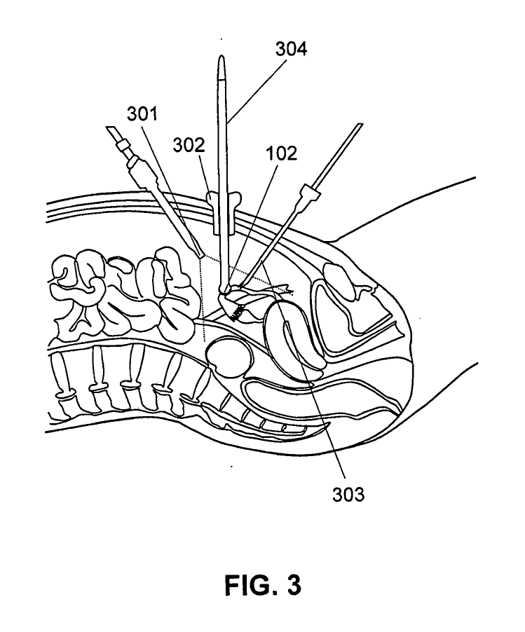

FIG. 3 illustrates the use of laparoscopic tactile imaging probe during a pelvic floor surgery. During a laparoscopic surgery a laparoscopic video camera becomes a surgeon's eyes, since the surgeon uses the image from the laparoscope 301 with video camera positioned inside the patient's body to perform the procedure. The laparoscope 301 has a light source to light the internal organs and tissue surfaces (see two doted lines coming from the laparoscope 301). A tissue dissector 303 is used to cut the tissue in a region of interest. A tactile imaging probe 304 is inserted into the body cavity through an incision or a trocar port 302 as shown in FIG. 3. A region of interest identified by the surgeon is investigated (scanned) by the scanhead with the tactile imaging array 102 to acquire a tactile feedback image in real time to assist in surgical dissection and decision making. The probe 304 can be used in traditional laparoscopic approaches being operated manually by probe handle and in robotic surgery systems where probe is operated by a robotic arm.

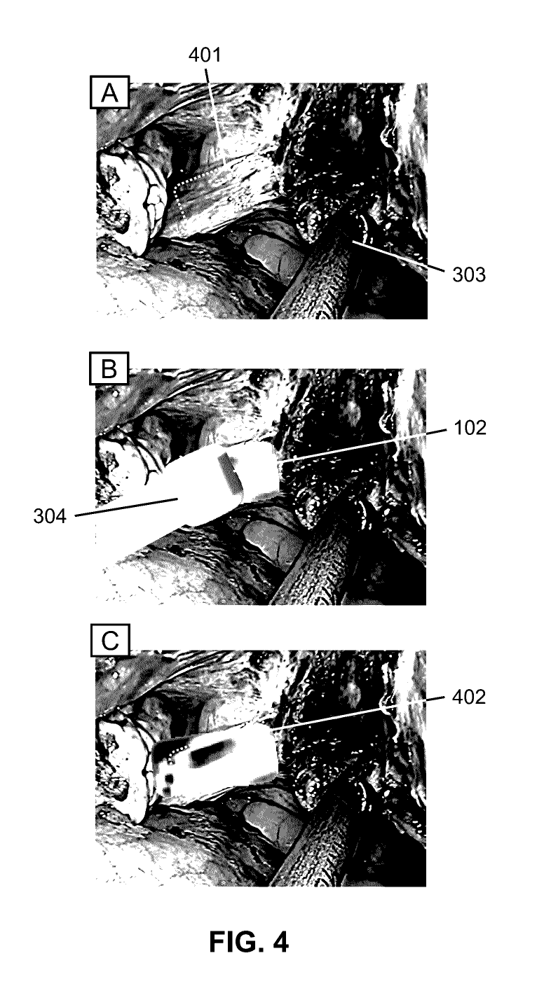

FIG. 4 illustrates the use of the tactile imaging probe 304 and the method for providing tactile feedback during laparoscopic surgery from the point of view of a surgeon. First of all, the surgeon selects the region of interest 401 within the laparoscopic video image (see FIG. 4A). The tissue dissector 303 is also within the video frame, but unmoved. On the next step (FIG. 4B), the surgeon applies the probe 304 with the tactile sensor array 102 to the selected region of interest 401 to scan this region. The scan includes application of a tissue deformation with the use of said probe by a human hand or a remotely controlled robot. This further includes tissue deformation as the result of applying a load to the tissue surface by said probe in orthogonal direction to said surface as well as a sliding by said probe along the tissue surface. After completion of the investigation (scanning) of the region of interest by the probe 304, the tactile feedback image 402 is imposed on the region of interest (see FIG. 4C). The two-dimensional tactile feedback image 402 is composed of the maximum values of the spatial gradients calculated within acquired three-dimensional tactile image of the region of interest. The spatial gradients directed orthogonally to soft tissue surface characterize distribution of tissue elasticity [Egorov V, van Raalte H, Lucente V, Sarvazyan A. Biomechanical characterization of the pelvic floor using tactile imaging. In: Biomechanics of the Female Pelvic Floor, Eds. Hoyte L, Damaser M S, 1st Editioin, Elsevier, Mar. 22, 2016: 317-348].

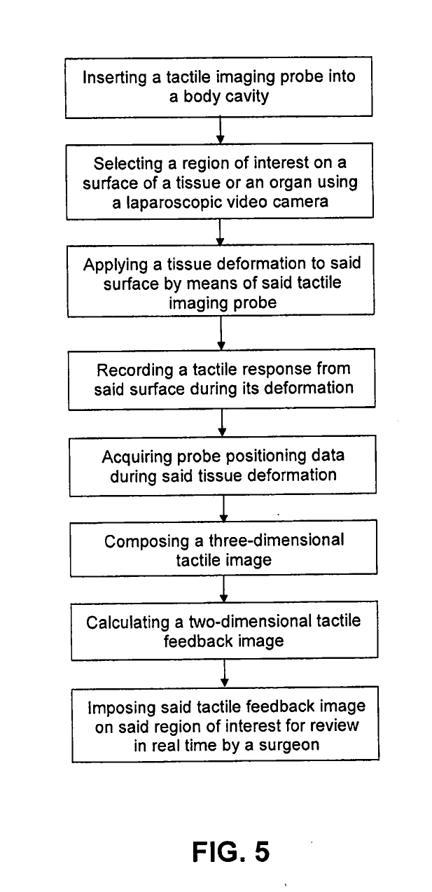

FIG. 5 is a flow chart illustrating one method for providing tactile feedback during laparoscopic surgery. Said method comprising the steps of:
inserting a tactile imaging probe into a body cavity;
selecting a region of interest on a surface of a tissue or an organ by means of an image projected by a laparoscopic video camera;
applying a tissue deformation to said surface by means of said tactile imaging probe;
recording a tactile response from said surface during its deformation,
acquiring probe positioning data during said tissue deformation;
composing a three-dimensional tactile image using said tactile response and said probe positioning data;
calculating a two-dimensional tactile feedback image using said three-dimensional tactile image; and
imposing said tactile feedback image on said region of interest for review in real time by a surgeon.

The probe can be inserted into a body cavity through a natural body orifice, a surgical incision or a trocar port.

The proposed method can be applied during lung, abdominal, gynecological, urological and urogynecological surgeries and said selecting a region of interest on a surface of a tissue or an organ can be completed in the course of these surgeries.

The tissue deformation can be the result of applying a load by said probe in orthogonal direction to said surface or can be the results of a sliding by said probe along said surface under applied load to the probe.

The step calculating a two-dimensional tactile feedback image further can include calculating tactile feedback image as a spatial gradient in orthogonal direction to said surface of said region of interest and the step imposing said tactile feedback image on said region of interest further can include the use of said tactile feedback image as a color scaled semi-transparent image.

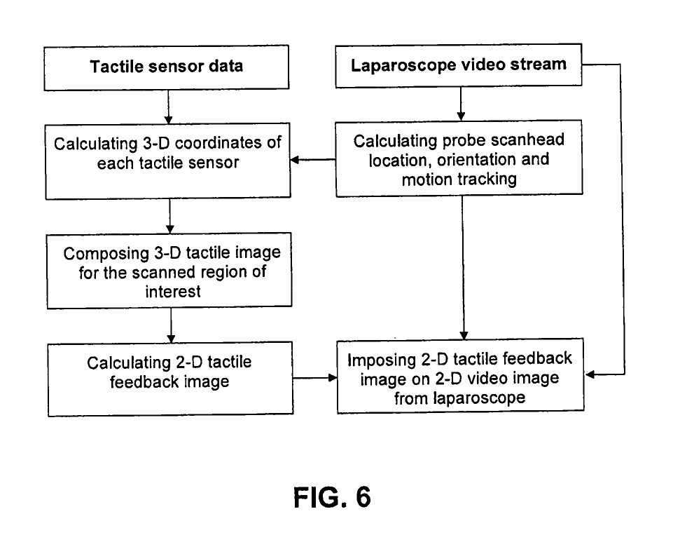

FIG. 6 is a flow chart illustrating details of data processing algorithm for providing tactile feedback for method shown in FIG. 5. The probe positioning data can be derived from a real time analysis of probe location by the probe visual markers within an image projected by a laparoscopic video camera. These positioning data further can be used for calculating 3-D coordinates of each tactile sensor, composing 3-D tactile image for the scanned region of interest and calculating 2-D tactile feedback image.

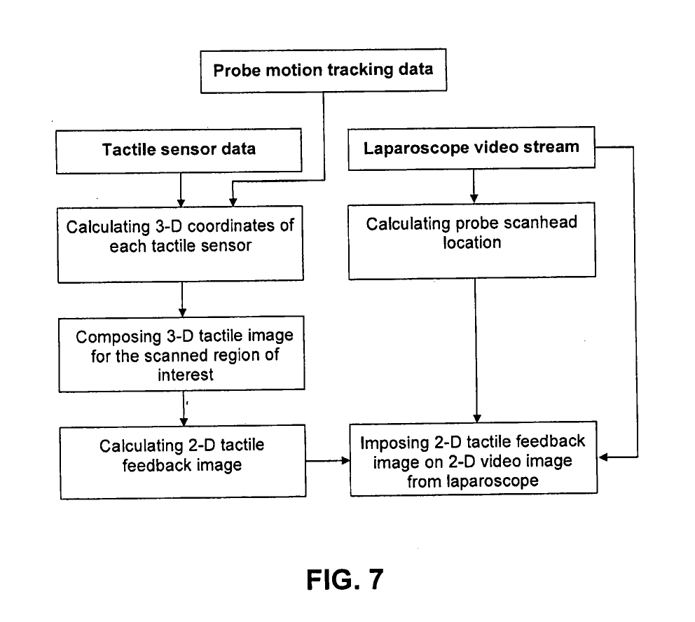

FIG. 7 is a flow chart illustrating another method for providing tactile feedback during laparoscopic surgery, herein probe motion tracking data are acquired from a motion tracking sensor and further used for calculating 3-D coordinates of each tactile sensor, composing 3-D tactile image for the scanned region of interest and calculating 2-D tactile feedback image.

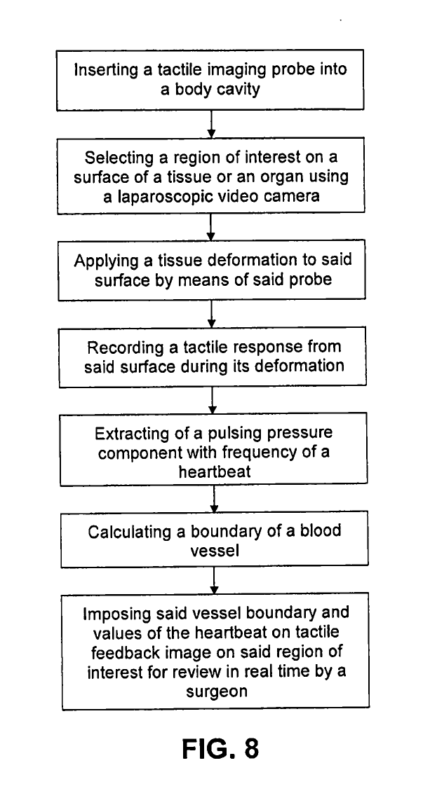

FIG. 8 is a flow chart illustrating another method for providing tactile feedback during laparoscopic surgery. Said method comprising the steps of:
inserting a tactile imaging probe into a body cavity;
selecting a region of interest on a surface of a tissue or an organ by means of an image projected by a laparoscopic video camera;
applying a tissue deformation to said surface by means of said probe;
extracting of a pulsing pressure component with frequency of a heartbeat;
calculating a boundary of a blood vessel;
imposing said vessel boundary and values of the heartbeat on tactile feedback image on said region of interest for review in real time by a surgeon.

The proposed method can be applied during lung, abdominal, gynecological, or urological surgeries and said selecting a region of interest on a surface of a tissue or an organ can be completed in the course of these surgeries.

The tissue deformation can be the result of applying a load by said probe in orthogonal direction to said surface or can be the results of a sliding by said probe along said surface under applied load to the probe.

The step extracting of a pulsing components by additional pressure sensor applied to a surface of a skin and can include synchronization with heartbeat pulse from easy assessable location on the surface a human body.

According to another embodiment, an image fusion is provided by combining tactile response recorded by tactile imaging probe with visual data acquired by laparoscopic video camera.

The herein described subject matter sometimes illustrates different components or elements contained within, or connected with, different other components or elements. It is to be understood that such depicted architectures are merely examples, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components.

Although the invention herein has been described with respect to particular embodiments, it is understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method for providing tactile feedback in laparoscopic surgery, said method comprising the steps of:
(i) inserting a tactile imaging probe into a body cavity;
(j) selecting a region of interest on a surface of a tissue or an organ by means of an image projected by a laparoscopic video camera;
(k) applying a tissue deformation to said surface by means of said tactile imaging probe;
(l) recording a tactile response from said surface during its deformation,
(m) acquiring probe positioning data during said tissue deformation;
(n) composing a three-dimensional tactile image using said tactile response and said probe positioning data;
(o) calculating a two-dimensional tactile feedback image using said three-dimensional tactile image; and
(p) imposing said tactile feedback image on said region of interest for review in real time by a surgeon.

2. The method as in claim 1, wherein step (c) further including said applying tissue deformation with the use of said probe by a human hand.

3. The method as in claim 1, wherein step (c) further including said applying tissue deformation with the use of said probe by a remotely controlled robot.

4. The method as in claim 1, wherein step (c) further including said tissue deformation as the result of applying a load to said surface by said probe in orthogonal direction to said surface.

5. The method as in claim 1, wherein step (c) further including said tissue deformation as the result of applying a load to said surface by said probe and a sliding by said probe along said surface.

6. The method as in claim 1, wherein step (e) further including said probe positioning data provided by a motion tracking sensor incorporated into said probe.

7. The method as in claim 1, wherein step (e) further including said probe positioning data being derived from a real time analysis of said probe location within said image projected by said laparoscopic video camera.

8. The method as in claim 1, wherein step (g) further including said tactile feedback image being calculated as a spatial gradient in orthogonal direction to said surface of said region of interest.

9. A method for providing tactile feedback in laparoscopic surgery, said method comprising the steps of:
(a) inserting a tactile imaging probe into a body cavity;
(b) selecting a region of interest on a surface of a tissue or an organ by means of an image projected by a laparoscopic video camera;
(c) applying a tissue deformation to said surface by means of said probe;
(d) extracting of a pulsing pressure component with frequency of a heartbeat,
(e) calculating a boundary of a blood vessel;
(f) imposing said vessel boundary and values of the heartbeat on tactile feedback image on said region of interest for review in real time by a surgeon.

10. The method as in claim 9, wherein step (d) further includes synchronization with heartbeat pulse from easy assessable location on the surface a human body.

11. A probe for providing tactile feedback in laparoscopic surgery, said probe comprising:
a probe housing with a front portion suitably shaped for atraumatic insertion into a body cavity,
a plurality of tactile sensors forming together a tactile array, said plurality of tactile sensors are located over a cylinder surface of said front portion, said plurality of tactile sensors are configured to record a tactile response from selected region of interest on a surface of a tissue or an organ being under a deformation provided by said front portion, and
a visual marker over said probe housing for visual acquiring probe positioning data during said deformation by a laparoscopic video camera.

12. The probe as in claim 11 wherein said visual markers includes at least three color points at vertices of a triangular wherein one vertex is placed on a probe shaft within the visual detection by said laparoscopic video camera.
